(19) United States
(12) Patent Application Publication
(10) Pub. No.: US 2008/0243016 A1
(43) Pub. Date: Oct. 2, 2008

(54) Pulmonary Artery Pressure Signals And Methods of Using

(75) Inventors: Wangcai Liao, Shoreview, MN (US); Jeffrey E. Stahmann, Ramsey, MN (US); Abhi V. Chavan, Maple Grove, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/692,687

(22) Filed: Mar. 28, 2007

(51) Int. Cl. A61B 5/00; A61B 5/08

(52) U.S. Cl. 600/532; 600/300

(57) ABSTRACT

Embodiments of the invention are related to methods and systems for using a pulmonary artery pressure signal to detect and/or monitor physiological parameters, physiological status, and aspects of disorders and diseases, amongst other things. In an embodiment, the invention includes a method of measuring a pulmonary function parameter of a patient. In an embodiment, the invention includes a method of detecting a sleeping disorder. In an embodiment, the invention includes a method of tracking sleep characteristics of a patient. Other aspects and embodiments are provided herein.

### TECHNICAL FIELD

This disclosure relates generally to methods of using a pulmonary artery pressure signal and, more particularly, to using a pulmonary artery pressure signal to detect and/or monitor physiological parameters, physiological status, and aspects of disorders and diseases, amongst other things.

### BACKGROUND OF THE INVENTION

Cardiopulmonary diseases afflict millions of people each year. In particular, diseases of the heart remain the leading cause of death in the United States. Monitoring patients' physiological state is an important aspect in the diagnosis, management and treatment of various diseases and disorders, including cardiopulmonary diseases. For this reason, significant efforts have been directed at improving monitoring and detection technologies. In specific, significant efforts have been directed at improving monitoring and detection technologies for cardiopulmonary diseases and related diseases that affect cardiopulmonary parameters.

Implantable medical devices can be advantageous as monitoring devices because the monitoring can be performed as desired, without regard to the physical location of the patient. In addition, the use of implantable medical devices for patient monitoring eliminates problems associated with patient compliance. However, many existing techniques for monitoring patients' physiological state cannot be implemented well in the context of implantable medical devices.

For at least these reasons, a need exists for methods of gathering physiological data regarding a patient with an implantable medical device. A need also exists for methods of detecting, diagnosing, predicting, and/or monitoring cardiopulmonary diseases and other conditions that affect cardiopulmonary parameters.

### SUMMARY OF THE INVENTION

Embodiments of the invention are related to methods and systems for using a pulmonary artery pressure signal to detect and/or monitor physiological parameters, physiological status, and/or aspects of disorders and diseases, amongst other things. In an embodiment, the invention includes a method of measuring a pulmonary function parameter of a patient including chronically implanting a pulmonary artery pressure sensor, obtaining a pulmonary artery pressure signal from the pressure sensor, and processing the signal to obtain the pulmonary function parameter.

In an embodiment, the invention includes a method of detecting a sleeping disorder including measuring a pulmonary artery pressure signal with a pressure sensor, and monitoring the pulmonary artery pressure signal to identify a breathing pattern indicative of a sleeping disorder.

In an embodiment, the invention includes a method of tracking sleep characteristics of a patient including monitoring a pulmonary artery pressure signal for a change to a cardiopulmonary parameter indicative of a sleep event, and

recording an occurrence of the sleep event if a change to the cardiopulmonary parameter indicative of the sleep event occurs.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a cross-sectional top view of the chest of a human showing the pulmonary artery in relation to the heart and the lungs.

FIG. 2 is a flowchart of a method for measuring a pulmonary function parameter.

FIG. 3 is a graph of an idealized pulmonary artery pressure signal and a respiration signal derived from the pulmonary artery pressure signal.

FIG. 4 is a graph showing an idealized respiration signal during normal breathing and during a forced expiration maneuver.

FIG. 5 is a graph showing an idealized respiration signal during normal breathing followed by forced inspiration and forced expiration.

FIG. 6 is a flow chart illustrating an embodiment of a method for detecting a disease or disorder.

FIG. 7 is a graph of an idealized respiration signal associated with a normal breathing pattern in comparison with an idealized respiration signal associated with a rapid and shallow breathing pattern.

FIG. 8 is a graph of an idealized respiration signal consistent with a pulmonary embolism.

FIG. 9 is a graph of an idealized respiration signal illustrating the effects of pulmonary arteriovenous malformation (PAVM).

FIG. 10 is a graph of an idealized respiration signal illustrating rapid expiration.

FIG. 11 is a graph of an idealized respiration signal illustrating the effects of an asthma attack.

FIG. 12 is a flowchart illustrating an embodiment of a method for detecting a disorder affecting airflow.

FIG. 13 is a graph of an idealized respiration signal showing apnea.

FIG. 14 is a graph of an idealized respiration signal showing hypopnea.

FIG. 15 is a graph of an idealized respiration signal showing Cheyne-Stokes respiration.

FIG. 16 is a flowchart illustrating a closed loop method for automatically adjusting the pressure of air delivered from an airway therapy device.

FIG. 17 is a flowchart illustrating a method for titrating air pressure delivered by an airway therapy device.

FIG. 18 is a flowchart illustrating a method for tracking sleep characteristics of a patient.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Monitoring a patient's physiological condition is an important aspect in the diagnosis, management and treatment of various diseases. One approach to monitoring the physiological state of patients is the use of an implantable medical device that can detect physiological conditions. The use of an implantable medical device as a monitoring device can be advantageous because the monitoring can be performed as frequently as desired, without regard to the physical location of the patient. In addition, patient monitoring with an implanted medical device eliminates or reduces problems associated with patient compliance.

One aspect of physiological status is the pressure of fluids, such as blood, at various points in the vasculature of a patient. Frequently, blood pressure is indirectly estimated based on readings taken by care providers during clinical visits using a sphygmomanometer. During sphygmomanometry, typically, an occluding cuff is inflated to a pressure level above arterial pressure as indicated by obliteration of the pulse. Then, the cuff is gradually deflated and the pressures are noted at which sounds produced by the arterial pulse waves (Korotkoff sounds) appear and disappear again as flow through the artery resumes. While sphygmomanometry is minimally invasive, it is less than ideal because of limited accuracy and reproducibility and because of its inability to measure blood pressure in remote embedded blood vessels within the circulatory system where pressure is different than the normal left sided systemic blood pressure. As such, direct measurement of fluid pressure within various parts of the heart and lungs is beneficial for purposes of both diagnosis and treatment.

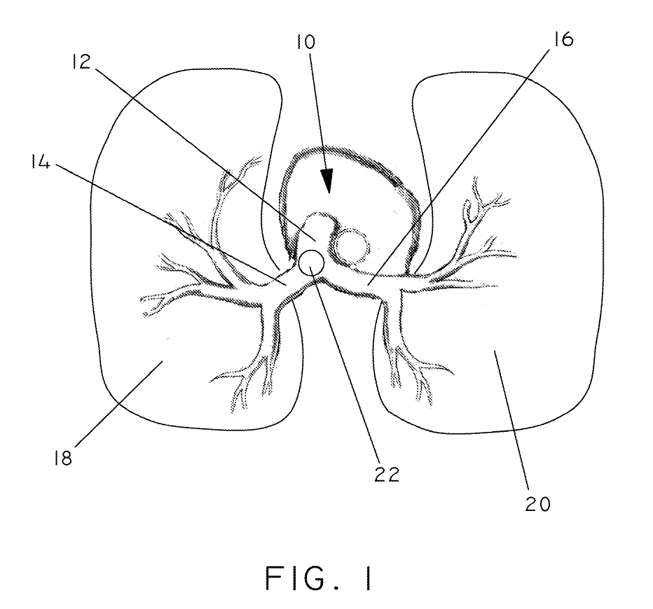

The pulmonary artery is one place where fluid pressure can be measured in order to provide cardiopulmonary status information to the clinician. While the vasculature commonly referred to as the “pulmonary artery” includes the pulmonary trunk (or main pulmonary artery) and the right and left pulmonary arteries, “pulmonary artery” is used in this invention to mean any artery supplying blood to the lungs. FIG. 1 shows a cross-sectional top view of the heart and parts of the pulmonary artery in a human. The pulmonary trunk 12 begins at the base of the right ventricle 10 and extends for approximately 2 inches in length before branching into the left pulmonary artery 14 and right pulmonary artery 16, which deliver deoxygenated blood to the left lung 18 and right lung 20 respectively. A pressure sensor 22 can be disposed within, or adjacent to, the pulmonary artery in order to generate a signal corresponding to pulmonary artery pressure. The pressure sensor can include any type of sensor, for example an electrical, mechanical, or optical sensor, that generates a signal in response to local pressure. By way of example, the pressure sensor can include devices such as those described in U.S. Pat. No. 6,237,398, the contents of which are herein incorporated by reference. The pressure sensor can be chronically implanted. The term “chronically implanted” as used herein with respect to a medical device shall refer to those medical devices that are implanted within an organism that are intended to remain implanted long-term, such as for a period of time lasting for months or years. Examples of chronically implanted medical devices include stents and pacemakers. Devices can be chronically implanted using standard surgical techniques.

Pulmonary artery pressure can be a useful indication of a patient's condition both directly and indirectly. For example, pulmonary artery pressure is useful because many diseases can result in elevated pulmonary artery pressure and therefore can be detected by monitoring pulmonary artery pressure. Pulmonary artery pressure is also useful because it is related to the pressure in other parts of the vasculature. For example, pulmonary artery pressure is related to the pressure in the left ventricle. Specifically, the pulmonary artery end-diastolic pressure (PAEDP) can be used to estimate left ventricle end-diastolic pressure (LVEDP), which is an important parameter of cardiopulmonary status. Left ventricle end-diastolic pressure (LVEDP) can also be referred to as left ventricle filling pressure or left ventricle pre-load. At the end of expiration during the respiratory cycle, intrathoracic pressure has little impact on pulmonary artery pressure. Therefore, LVEDP can be estimated based on the PAEDP as measured at the end of expiration.

The range of hemodynamic information that can be obtained with a coronary artery pressure sensor can include many different parameters. By way of example, hemodynamic information that can be obtained with a coronary artery pressure sensor can include the systolic pulmonary artery pressure at end-expiration, the diastolic pulmonary artery pressure at end-expiration, the mean pulmonary artery pressure, the systolic duration, the diastolic duration, the slew rate of the pulmonary artery pressure, dP/dt, the amplitude, duration and timing of the dicrotic notch, heart rate, and heart rate variability, among others.

In addition to this hemodynamic information, it has been discovered that a pulmonary artery pressure signal can be processed in order to determine or estimate one or more parameters of pulmonary function (“pulmonary function parameters”). Pulmonary artery pressure is modulated by intrathoracic pressure, which changes with inspiration and expiration. Specifically, intrathoracic pressure is increased during expiration and decreased during inspiration. The relationship between pulmonary artery pressure and intrathoracic pressure can be used in a method in order to derive one or more pulmonary function parameters. In an embodiment, the invention includes a method of measuring a pulmonary function parameter of a patient using a pulmonary artery pressure signal.

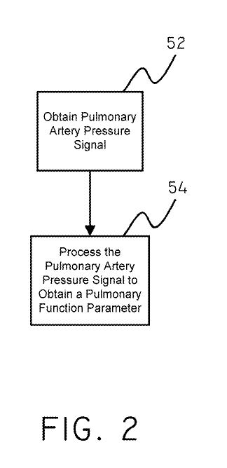

By way of example, FIG. 2 shows a flowchart of a method embodiment for measuring a pulmonary function parameter. First, a pulmonary artery pressure signal is obtained 52 from a pressure sensor that is disposed in or near the pulmonary artery. Next, the pulmonary artery pressure signal is processed 54 in order to obtain a pulmonary function parameter. Processing of the pulmonary artery pressure signal can include various steps, some of which are described more fully below. While not intending to be bound by theory, because of the anatomical relationship between the pulmonary artery and the lungs, it is believed that there are advantages to deriving pulmonary parameters from a pulmonary artery pressure signal in contrast to pressure signals representing the pressure in other parts of the vasculature. For example, such advantages can include accuracy, ease of calculation, and the like.

Many different pulmonary function parameters can be calculated or estimated by processing a pulmonary artery pressure signal. For example, such pulmonary function parameters can include respiration waveforms (both inspiration and expiration), respiration rate, respiratory rate variability, respiratory excursion, breath interval, inspiration slope, expiration slope, tidal volume, relative tidal volume, minute ventilation, relative minute ventilation, pulmonary vascular resistance, relative pulmonary vascular resistance, forced expiration volume in one minute (FEV1), relative forced expiration volume in one minute (FEV1), forced vital capacity (FVC), relative forced vital capacity (FVC), ratio of FEV1 to FVC, total lung capacity (TLC), relative total lung capacity (TLC), and the like.

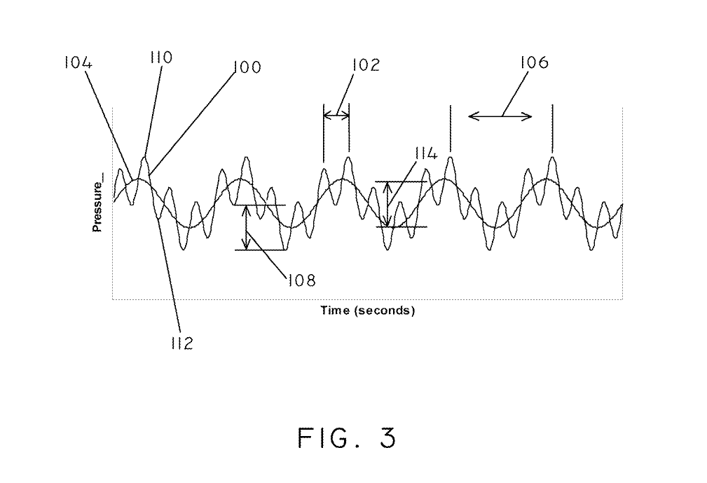

Referring now to FIG. 3, a graph of an idealized pulmonary artery pressure signal 100 is illustrated. The pressure signal is a series of peaks 110 and valleys 112, where each peak 110 corresponds to the maximum systolic pressure during the cardiac cycle and each valley 112 corresponds to the minimum diastolic pressure during the cardiac cycle. The time for each cardiac cycle can be measured simply by measuring the amount of time 102 in between each successive peak (or valley) of the pressure signal. As can be seen in FIG. 3, the pressure peaks and valleys cyclically rise and fall with time as a result of changes in intrathoracic pressure during inspiration and expiration. The amount of the difference in pressure between inspiration and expiration can be referred to as the respiratory excursion 108. When processing a pulmonary artery pressure signal, the respiratory excursion 108 can be calculated by measuring the maximum difference in pressure between successive inspiration and expiration at the same relative point in the cardiac contraction cycle, typically during systole or diastole.

Respiration line 104 illustrates a roughly sinusoidal respiratory artifact that is superposed on pulmonary artery pressure and is caused by changes in intrathoracic pressure during the respiration cycle. Respiration line 104 can be calculated based on the pulmonary artery pressure signal 100 using various techniques. For example, the respiration line 104 can be calculated by tracking the fluctuation of the pulmonary artery pressure peaks over time. As another example, the respiration line 104 can be calculated by tracking the fluctuation of the pulmonary artery pressure valleys over time. In some embodiments, filtering can be used to separate the respiration and cardiac components of pulmonary artery pressure signal 100. For example, a lowpass filter with a cutoff frequency of approximately 0.5 Hz would substantially pass the respiratory component of the pulmonary artery pressure signal 100, thus creating respiration line 104, while significantly attenuating the cardiac component. Further, filtering pulmonary artery pressure signal 100 with a high pass filter with a cutoff frequency of approximately 0.75 Hz would substantially pass the cardiac component of the pulmonary artery pressure signal 100, while significantly attenuating the respiratory component. To improve respiratory and cardiac signal separation, the cutoff frequencies of the lowpass and highpass filters may be decreased and increased with decreasing and increasing respiratory and/or cardiac rates respectively.

Respiration line 104 (or the “respiration signal”) can, in turn, be used to calculate many different pulmonary parameters. The contours of the respiration line 104 over time can be referred to as the respiration waveform. The slope of the respiration line 104 as it is rising (as during expiration) and as it is falling (as during inspiration) can be tracked and recorded. In this manner, both the inspiration slope and expiration slope can be calculated.

The time for each cycle of respiration (both expiration and inspiration) can be determined by measuring the amount of time 106 in between successive peaks (or valleys) of the respiration line 104. The amount of time between successive peaks can be referred to as the breath interval. The respiration rate can then be calculated simply by dividing the desired time period, such as one minute, by the breath interval (time for each cycle of respiration). For example, if the time for each cycle is found to be two seconds, then the respiration rate would be thirty breaths per minute. The respiration rate can be calculated in real-time. The respiration rate can also be recorded and tracked over a period of time. In this manner, respiratory rate variability can be calculated.

The amplitude 114 of the respiration line 104 corresponds to how deep or shallow the breathing of the patient is. The term “tidal volume” refers to the amount of air breathed in or out during normal respiration. As such, the amplitude 114 of the respiration line 104 can be used to estimate relative tidal volume. The tidal volume can be estimated in real time and/or recorded over a period of time. By way of example, in an embodiment, a baseline value for the net amplitude of the respiration line 104 from peak to valley can be established for a given patient and then measurements in real time can be compared with the baseline value to derive a relative tidal volume value. This method can be used to assess whether the tidal volume of the patient is increasing or decreasing over time.

In some embodiments, the baseline value can simply be based on historical data derived from the pulmonary artery pressure signal. In other embodiments, the baseline value can be calibrated by using data from another instrument. As one example, during a calibration procedure, a patient can be prompted to blow into an air flow meter while the pulmonary artery pressure signal is being recorded. Data from the air flow meter can be used to accurately calculate the actual tidal volume. The recorded pulmonary artery signal can then be calibrated to the actual tidal volume as indicated by the air flow meter. Estimates of the actual tidal volume can be made in real time by applying this calibration data to the pulmonary artery pressure signal.

The relative tidal volume can in turn be used to estimate other parameters. For example, minute ventilation is defined as the tidal volume multiplied by the respiration rate (in breaths/minute). As such, the relative tidal volume, as calculated above, can be multiplied by the respiration rate in order to derive a relative minute ventilation value.

Pulmonary vascular resistance (PVR) refers to the resistance offered by the vasculature of the lungs to the flow of blood. The units for measuring vascular resistance are dyn-s/cm⁵. PVR can be estimated using a pulmonary artery pressure signal by the formula: PVR=((mean pulmonary artery pressure−end-diastolic pulmonary artery pressure)/cardiac output)×80, where pressures are in mmHg and cardiac output is measured in liters per minute. Conventionally, pulmonary capillary wedge pressure is used in the formula instead of end-diastolic pulmonary artery pressure. However, it is widely accepted that end-diastolic pulmonary artery pressure can be used as an estimation of pulmonary capillary wedge pressure.

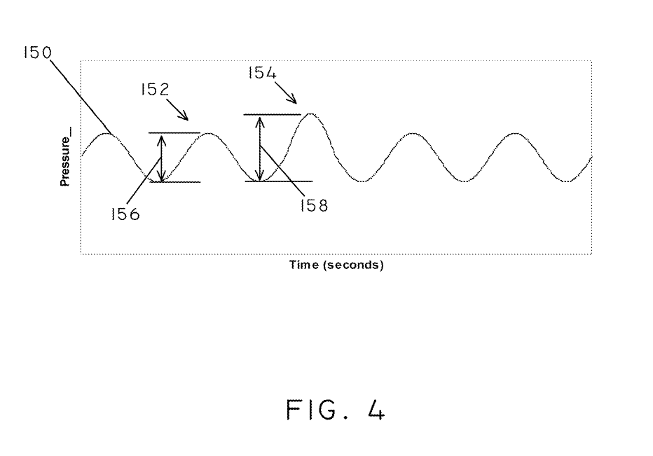

Forced expiratory volume (FEV₁) refers to the amount of air that a patient can forcibly exhale in one second. This value can be estimated using a pulmonary artery pressure signal in various ways. For example, in some embodiments, a patient can be given a cue indicating that they should forcibly blow out as much air as possible. The pulmonary artery pressure signal can be captured during this forcible expiration and then processed to provide an estimation of the volume expelled during a one second span of time. For example, the pulmonary artery pressure signal can be processed into a respiration signal (such as respiration line 104 in FIG. 3). The volume can then be determined based on further processing of the respiration signal. For example, referring now to FIG. 4, a graph is shown of a respiration signal 150 during normal breathing 152 and during forced expiration 154. The forced expiration amplitude 158 of the respiration signal 150 can be tracked and compared with the normal breathing amplitude 156 of the respiration signal 150. Then, based on the relationship of the normal breathing amplitude 156 to tidal volume an estimate of the volume expelled in one second during forced expiration 154 can be made. It will be appreciated that the value for FEV₁ can be either relative or absolute. For example, the value of FEV₁ can be in relation to the historical value of FEV₁ for the patient. In other embodiments, the FEV₁ can be absolute if, for example, the respiration signal 150 is calibrated after implantation against a reference value. For example, a patient with an implanted device generating a pulmonary artery pressure signal could be evaluated using a spirometer. Data from the spirometer can then be used to calibrate the respiration signal 150.

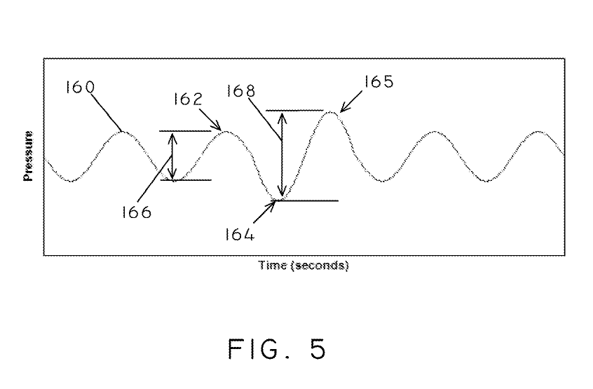

Forced vital capacity (FVC) refers to the total volume of air that a patient can forcibly blow out after full inspiration. This value can be estimated using a pulmonary artery pressure signal in various ways. For example, in some embodiments, a patient can be given a cue indicating that they should breathe in as much air as they can and then forcibly exhale as much air as possible. The pulmonary artery pressure signal can be captured during this forcible expiration and then processed to provide an estimation of the total volume of air expired during a one second span of time. For example, the pulmonary artery pressure signal can be processed into a signal indicative of respiration (such as respiration line 104 in FIG. 3). The volume can then be determined based on processing of the signal indicative of respiration. For example, referring now to FIG. 5, a graph is shown of a respiration signal 160 during normal breathing 162 and during forced inspiration 164 followed by forced expiration 165. The vital capacity amplitude 168 of the respiration signal 160 can be tracked and compared with the normal breathing amplitude 166 of the respiration signal 160. Then, based on the relationship of the normal breathing amplitude 166 to tidal volume, an estimation of the vital capacity volume can be made.

It will be appreciated that the value for FVC can be either relative or absolute. For example, the value of FVC could be in relation to the historical FVC of the patient. In other embodiments, the FVC can be absolute if, for example, the respiration signal is calibrated after implantation against a reference value. For example, a patient with an implanted device generating a pulmonary artery pressure signal could be evaluated using a spirometer. Data from the spirometer can then be used to calibrate the respiration signal.

The ratio of FEV₁/FVC can serve as a useful diagnostic measure. In healthy adults, this ratio is approximately 0.75 to 0.80. In some embodiments, the ratio of FEV₁ to FVC can be calculated by dividing FEV₁ (calculated as described above) by FVC (calculated as described above). The ratio of FEV₁/FVC can then be used further. For example, this ratio can be stored and then output to a care provider.

Total lung capacity (TLC) refers to the volume of gas contained in the lung at the end of maximal inspiration. TLC can also be referred to as the peak inspiratory volume. TLC is equal to the sum of forced vital capacity (FVC) plus residual volume. Where a patient forcibly blows out after full inspiration, the point of maximum inspiration defines the TLC, and the point of maximum expiration defines the residual volume. By convention, the volume between maximum inspiration and maximum expiration is the forced vital capacity (FVC), as described above. The residual volume is a value that can be calibrated using conventional techniques for measuring residual volume. As such, TLC can be estimated using pulmonary artery pressure signal by determining FVC and using a calibrated value for residual volume.

A pulmonary artery pressure signal can also be used to evaluate, detect, monitor, predict and/or identify various disease states that impact pulmonary function parameters. Cardiopulmonary diseases can include those diseases that are related to pathological structural pulmonary changes (“structural pulmonary diseases”). Pathological structural pulmonary changes can include changes to the tissue, structure, or fluid in or around the lung. In some embodiments, the invention includes a method for detecting pulmonary symptoms of a disorder including obtaining a pulmonary artery pressure signal from a pressure sensor and monitoring the pulmonary artery pressure signal to identify a change in the signal over a baseline value. In some embodiments, the invention includes a method for detecting a pathological change to a tissue, structure, or fluid volume in or around the lung, the method including establishing a baseline signal pulmonary artery pressure signal with a pressure sensor and monitoring the pulmonary artery pressure signal to identify a change in the pulmonary artery pressure signal compared to the baseline signal.

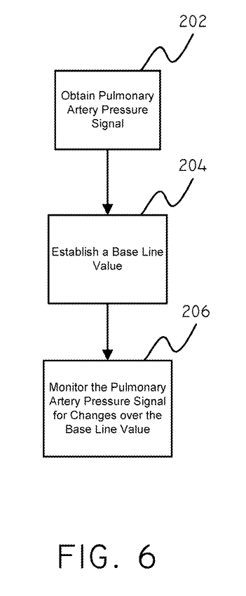

Referring now to FIG. 6, a flowchart is shown of a method for detecting a disorder or disease, such as a disorder or disease including a pathological structural change. A pulmonary artery pressure signal is obtained 202 from a pressure sensor that is disposed in or near the pulmonary artery. A baseline value for the pulmonary artery pressure signal is then established 204. The pulmonary artery pressure signal is then monitored 206 to identify changes with respect to the baseline value. In some embodiments, the pulmonary artery pressure signal is also converted to a respiration signal.

Specific examples of structural pulmonary diseases can include pulmonary edema, pulmonary embolism, pleural effusion, pulmonary arteriovenous malformation, combined obstructive pulmonary disease (COPD), asthma, and emphysema, amongst others. These diseases can affect various hemodynamic and/or pulmonary parameters. As described above, many hemodynamic and pulmonary parameters can be calculated or estimated based on a pulmonary artery pressure signal.

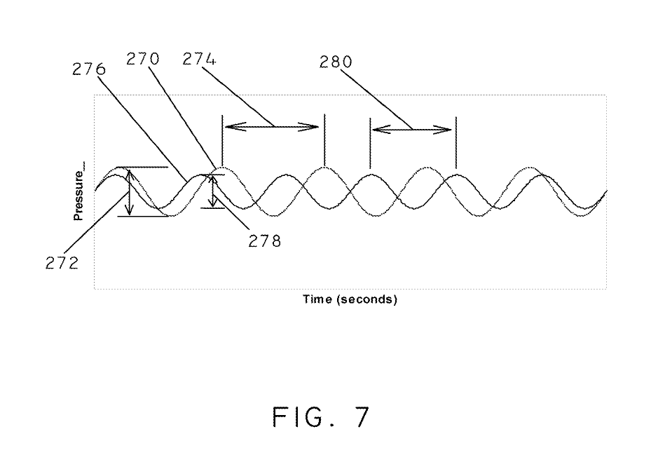

Pulmonary edema is a condition in which there is fluid accumulation in the lungs. Frequently, pulmonary edema is associated with heart failure. The accumulation of fluid in the lungs associated with pulmonary edema typically results a rapid and shallow (low tidal volume) breathing pattern. Monitoring of a pulmonary artery pressure signal can be used to identify this rapid and shallow breathing pattern. Specifically, a pulmonary artery pressure signal can be processed, as described above, in order to calculate and/or estimate both a breathing rate and a tidal volume. Values for breathing rate and tidal volume can then be evaluated to detect a breathing pattern consistent with pulmonary edema. Referring now to FIG. 7, a graph is shown illustrating a respiration signal associated with a normal breathing pattern 270 and a respiration signal associated with a rapid and shallow breathing pattern 276. The normal breathing pattern amplitude 272 is larger than the rapid and shallow pattern amplitude 278. In addition, the normal peak to peak distance 274 (indicative of the time for each respiration cycle) is larger than the rapid and shallow peak to peak distance 280.

In some embodiments, if pulmonary parameters are consistent with a diagnosis of pulmonary edema, the event can be flagged and logged and/or an alert can be generated. This alert can be transmitted to a care provider for further action. For example, the alert can be transmitted to a care provider during interrogation of the device, such as during an office visit. As a further example, the alert can be delivered to a care provider through an advanced patient management system such as the LATITUDE® patient management system, commercially available from Boston Scientific Corporation, Natick, Mass. Aspects of an exemplary advanced patient management system are described in U.S. Pat. No. 6,978,182, the contents of which are herein incorporated by reference. As pulmonary edema can be progressive condition, in some embodiments, monitoring can be performed over a period of time to monitor the severity of the condition. By way of example, data regarding pulmonary parameters can be stored by the system and then compared with data taken in real-time. In this manner, an indication of whether the condition is improving or worsening can be derived.

A pulmonary embolism is where a blood clot lodges in the lumen (open cavity) of a pulmonary artery, occluding the artery and causing dysfunction. Pulmonary emboli (clots) often originate in the deep leg veins and travel to the lungs through blood circulation. A pulmonary embolism can be manifested by a rapid and shallow (low tidal volume) breathing pattern and, in some cases, coughing. In addition, the pressure of blood in the pulmonary artery would be expected to rapidly rise in response to a pulmonary embolism. The specific degree to which pulmonary artery pressure would rise would depend on various factors including the size of the embolus and where the embolus is lodged in the pulmonary arterial vasculature.

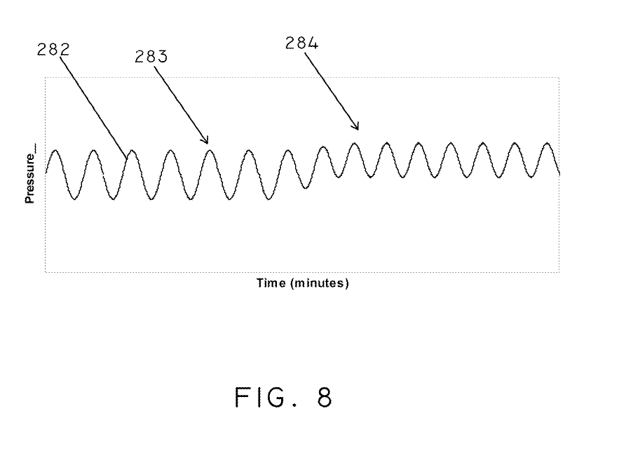

Monitoring of a pulmonary artery pressure signal can be used to identify a pulmonary embolism. Specifically, a pulmonary artery pressure signal can be monitored to identify a rapid and shallow breathing pattern. A pulmonary artery pressure signal can be processed, as described above, in order to calculate and/or estimate both a breathing rate and a tidal volume. Values for each of these pulmonary parameters can then be evaluated in order to detect a pulmonary embolism. Monitoring of a pulmonary artery pressure signal can also be used to identify coughing. Referring now to FIG. 8, an idealized graph of a respiration signal 282 over time is shown illustrating a breathing pattern that becomes rapid and shallow in conjunction with elevated pressure in the pulmonary artery. The respiration signal 282 changes from a normal respiration pattern 283 to an abnormal respiration pattern 284 characterized by reduced amplitude and increased frequency. In addition, the pressure is increased causing the respiration signal 282 to be shifted upward in the abnormal respiration pattern 284.

In some embodiments, if pulmonary parameters are consistent with a diagnosis of a pulmonary embolism, the event can be flagged and logged and/or an alert can be generated by the system. This alert can be transmitted to a care provider for further action, such as through an advanced patient management system. Because a pulmonary embolism is usually caused by a blood clot lodging in a major pulmonary artery, the symptoms associated with a pulmonary embolism frequently appear quickly. As such, in some embodiments, the rapid onset of a rapid and shallow breathing pattern can be interpreted by the system as an indication of a pulmonary embolism.

Pleural effusion refers to a condition involving the buildup of fluid between the membranes that line the lungs and chest cavity (the pleura), causing compression of the lungs, which can lead to breathing difficulty. Pleural effusion can be manifested as a rapid and shallow (low tidal volume) breathing pattern. Monitoring of a pulmonary artery pressure signal can be used to identify this rapid and shallow breathing pattern (such as the rapid and shallow pattern illustrated in FIG. 7). Specifically, a pulmonary artery pressure signal can be processed, as described above, in order to calculate and/or estimate both a breathing rate and a tidal volume. Values for breathing rate and tidal volume can then be evaluated to detect a breathing pattern consistent with pleural effusion.

In some embodiments, if pulmonary parameters are consistent with a diagnosis of pleural effusion, the event can be flagged and logged and/or an alert can be generated by the system. This alert can be transmitted to a care provider for further action, such as through an advanced patient management system. As pleural effusion can be progressive condition, in some embodiments, monitoring can be performed over a period of time to monitor the severity of the condition. By way of example, data regarding pulmonary parameters can be stored by the system and then compared with data taken in real-time. In this manner, an indication of whether the condition is improving or worsening can be derived.

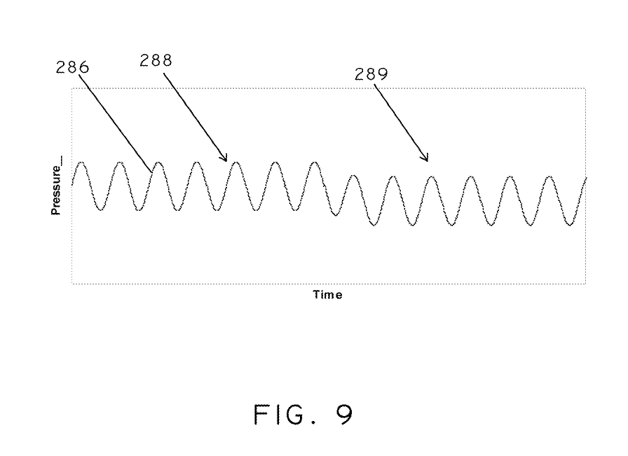

Pulmonary arteriovenous malformation (PAVM) refers to a malformation of the vasculature resulting in direct intrapulmonary connections between the pulmonary arteries and veins without an intervening capillary bed. This causes a right to left shunt with peripheral arterial oxygen desaturation. PAVM may result in lowered blood pressure within the pulmonary artery as the resistance to blood flow normally generated by an intervening capillary bed is reduced. Monitoring of a pulmonary artery pressure signal can be used to identify a reduced amount of pressure within the pulmonary artery. Referring now to FIG. 9, an idealized graph of a respiration signal 286 over time is shown illustrating a drop in pressure in the pulmonary artery. The respiration signal 286 changes from a first state 288 to a second state 289 that is characterized by reduced pressure. This change may occur over a time period of minutes to weeks. In some embodiments, if the pulmonary artery pressure signal is consistent with a diagnosis of PAVM, the event can be flagged and logged and/or an alert can be generated by the system. This alert can be transmitted to a care provider for further action, such as through an advanced patient management system.

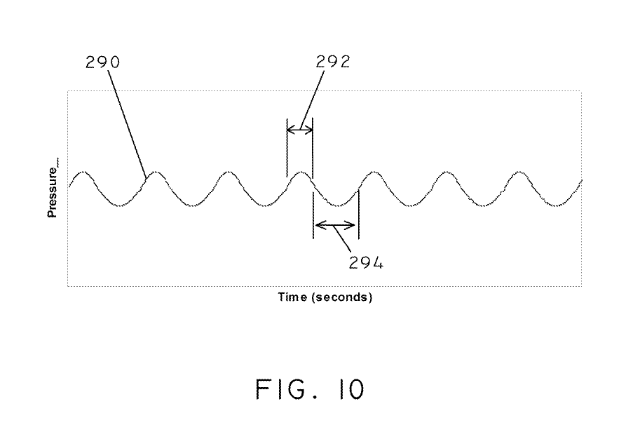

Chronic obstructive pulmonary disease (COPD) is a disease state characterized by airflow obstruction caused by chronic bronchitis, emphysema, or both. Emphysema is a pathological condition of the lungs marked by an abnormal increase in the size of the air spaces, resulting in labored breathing and an increased susceptibility to infection. It can be caused by irreversible expansion of the alveoli or by the destruction of alveolar walls. COPD and emphysema can be manifested by a breathing pattern that is both rapid and shallow, in addition to a shortened expiration time. Referring now to FIG. 10, a graph of a respiration signal 290 is shown illustrating a rapid and shallow breathing pattern with a shortened expiration time. Inspiration time is reflected by time period 294 corresponding to the lower pressure portion of the respiration cycle and expiration time is reflected by time period 292 corresponding to the higher pressure portion of the respiration cycle. In this case, time period 292 is shorter than time period 294 reflecting a shortened expiration time.

Monitoring of a pulmonary artery pressure signal can be used to identify a rapid and shallow breathing pattern. Specifically, a pulmonary artery pressure signal can be processed, as described above, in order to calculate and/or estimate both a breathing rate and a tidal volume. Values for breathing rate and tidal volume can then be evaluated to detect a breathing pattern consistent with COPD and/or emphysema. In addition, monitoring of a pulmonary artery pressure signal can be used to estimate expiration time. Expiration time can be used in conjunction with the rapid and shallow breathing pattern to suggest a diagnosis of COPD or emphysema. In some embodiments, if pulmonary parameters are consistent with a diagnosis of COPD or emphysema, the event can be flagged and logged and/or an alert can be generated by the system. This alert can be transmitted to a care provider for further action, such as through an advanced patient management system. As COPD and emphysema can be progressive, chronic conditions, in some embodiments, monitoring can be performed over a period of time to monitor the severity of the condition. By way of example, data regarding pulmonary parameters can be stored by the system and then compared with data taken in real-time. In this manner, an indication of whether the condition is improving or worsening can be derived.

Asthma is a chronic respiratory disease, often arising from allergies, that is characterized by sudden recurring attacks of labored breathing, chest constriction, and coughing. Asthma can be manifested by coughing and/or reduced peak air flow. In some cases, asthmas can be manifested by lengthened expiration time.

Monitoring of a pulmonary artery pressure signal can be used to identify symptoms consistent with asthma including coughing, reduced peak air flow, and/or lengthened expiration time. Specifically, a pulmonary artery pressure signal can be processed, as described above, in order to determine whether or not a patient is coughing. Coughing can be manifested as one or more sharp rises in pressure. In addition, a pulmonary artery pressure signal can be processed, as described above, in order to estimate peak air flow and this can be compared with stored values for peak air flow in order to determine whether or not there has been a reduction. Finally, the pulmonary artery pressure signal can be processed, as described above, in order to estimate expiration time. Expiration time can be compared with inspiration time in order to determine whether expiration time is lengthened. The presence of one or more of these symptoms can be indicative of asthma.

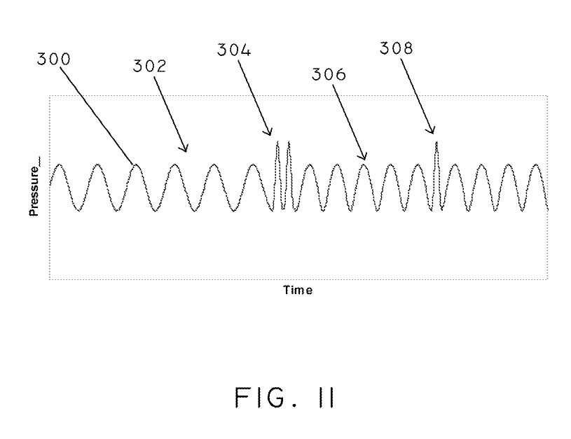

Referring now to FIG. 11, a graph of a respiration signal 300 is shown illustrating a rapid onset of coughing and lengthened expiration time relative to inspiration time. The respiration signal 300 follows a normal pattern 302 until two sharp increases 304 in pressure are detected consistent with coughing. The respiration signal 300 then follows a pattern 306 reflecting lengthened expiration time relative to inspiration time, along with another spike in pressure 308 consistent with a cough. In some embodiments, if symptoms are detected that are consistent with a diagnosis of asthma or an asthma attack, the event can be flagged and logged and/or an alert can be generated by the system. In some embodiments, the alert can be conveyed to a care provider, such as through an advanced patient management system. In some embodiments, the presence of symptoms consistent with an asthma attack can be used to initiate administration of a therapeutic agent that can counteract the effects of the asthma attack.

It will be appreciated that detection of various abnormalities in pulmonary function may not always be specific enough to provide for differential diagnosis of the condition. However, detection of pulmonary symptoms is still of significant value even in the absence of differential diagnosis. For example, pulmonary symptoms such as rapid and shallow breathing, coughing, increases or decreases in pressure, and the like can be tracked and/or logged and later conveyed to a care provider. For example, detection of spikes in pressure characteristic of coughing can be logged and then later provided to a care provider along with a time stamp of when they occurred in order to provide information about a patient's pulmonary function.

In some embodiments, the invention includes methods or detecting, trending, and/or predicting diseases, conditions, and symptoms associated with a permanent or temporary pathological change to airflow (“airway disorders”). By way of example, such diseases, conditions, and/or symptoms can include snoring, sleep apnea, hypopnea, hyperpnea, dyspnea, tachypnea, Cheyne-Stokes syndrome and the like.

Snoring refers to breathing during sleep with a rough hoarse noise due to vibration of the soft palate. Sleep apnea refers to a group of disorders in which breathing during sleep stops for at least ten seconds during sleep. Hypopnea refers to abnormally slow or shallow breathing. Hyperpnea refers to abnormally rapid or deep breathing. Dyspnea refers to difficult or labored breathing. Tachypnea refers to abnormally rapid breathing. Cheyne-Stokes syndrome (or Cheyne-Stokes respiration) is characterized by regularly alternating periods of apnea and hyperpnea. Because these disorders include effects on respiration, monitoring of respiration as calculated from a pulmonary artery pressure signal can provide useful information on the scope, severity, and/or progression of the disorder.

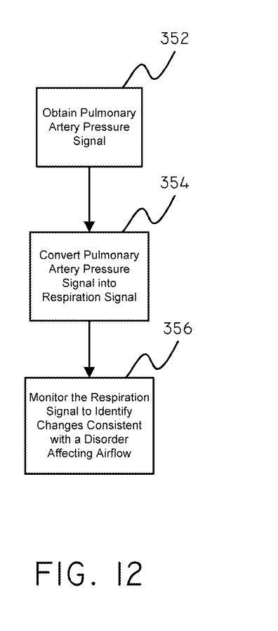

In an embodiment, the invention includes a method for detecting a disorder affecting airflow including obtaining a pulmonary artery pressure signal from a pressure sensor; and monitoring the pulmonary artery pressure signal to identify a respiration pattern consistent with the disorder. By way of example, referring now to FIG. 12 a flowchart is shown illustrating steps in an embodiment of a method for detecting a disorder affecting airflow. A pulmonary artery pressure signal is obtained 352 from a pressure sensor that is disposed in or near the pulmonary artery. In some embodiments, the pulmonary artery pressure signal is then converted into a respiration signal 354. Various techniques for converting a pulmonary artery pressure signal into a respiration signal are described above. Next, the respiration signal is monitored 356 to identify changes consistent with a disorder affecting airflow.

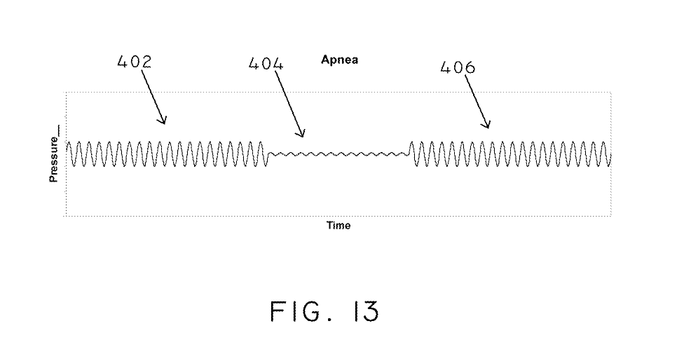

FIG. 13 shows a graph of a respiration signal over time as consistent with apnea. In this graph, breathing proceeds relatively normally for a period 402. Then apnea 404 occurs and breathing is interrupted. The interruption to breathing can last for varying lengths of time. In some instances, the interruption lasts for a period of time equal to or greater than ten seconds. Then, after arousal of the patient, normal breathing resumes for another period 406. This cycle can be repeated up to hundreds of times per night.

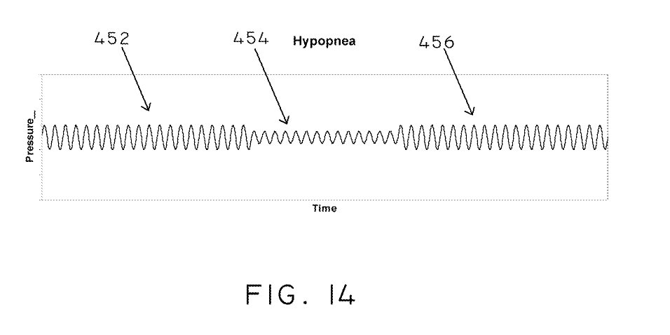

FIG. 14 shows a graph of a respiration signal over time as consistent with hypopnea. In this graph, breathing proceeds relatively normally for a period 452. Then hypopnea 454 occurs and breathing becomes very shallow. This shallow breathing can occur for varying lengths of time. In some instances, the shallow breathing occurs for a period of time equal to or greater than ten seconds. Then, after arousal of the patient, normal breathing resumes for another period 456. This cycle can be repeated up to hundreds of times per night.

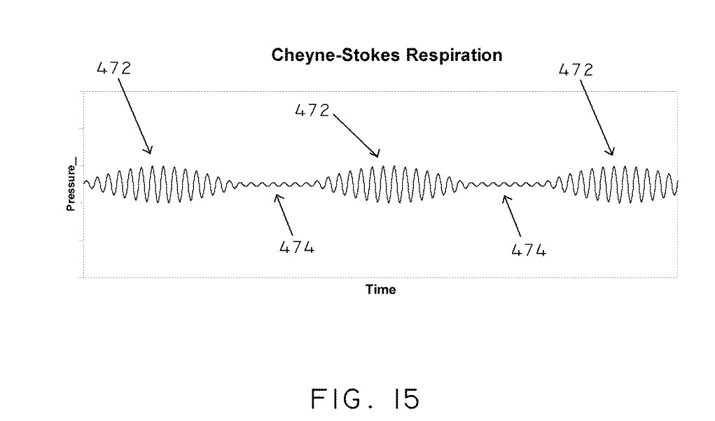

FIG. 15 shows a graph of tidal volume versus time as consistent with Cheynes-Stokes respiration. Cheynes-Stokes respiration is characterized by a plurality of periods 472 where respiration is first increasing and then decreasing in amplitude or tidal volume (sometimes referred to as crescendos and decrescendos), interrupted by a plurality of central apneas 474. Embodiments of the invention can be used to identify breathing patterns consistent with hypernea, dyspnea, hypopnea, apnea, tachypnea, and/or Cheyne-Stokes respiration. When such a breathing pattern is identified, the event can be flagged and logged and/or reported to a care provider.

Therapies that can be used to treat airway disorders can include continuous positive airway pressure (CPAP), bi-level positive airway pressure (BiPAP), electrical diaphragm stimulation (EDS), and the like. In some embodiments, the invention can include methods of initiating or modifying respiratory therapy based on the occurrence and/or degree of airway dysfunction. By way of example, a method can include continuously collecting cardiopulmonary information as feedback on the application of therapy and then adjusting the internal or external respiratory therapy as indicated.

As a specific example, continuous positive airway pressure (CPAP) is frequently used to treat obstructive sleep apneas and involves the delivery of compressed air into the nasal passage of a patient, typically via a mask. The CPAP machine blows air at a prescribed pressure (the “titrated pressure”). The necessary pressure is usually determined by a physician after review of an overnight sleep study in a sleep laboratory. The titrated pressure is the pressure of air at which most (if not all) apneas and hypopneas have been prevented, and it is usually measured in centimeters of water (cm/H₂O). CPAP machine generally can deliver pressures between 4 and 30 cm. CPAP is believed to work by pneumatically splinting the upper airway, decreasing the severity of obstruction.

Although CPAP has no serious side effects in most patients with sleep apnea, there are several minor pressure related side effects that reduce patient compliance and quality of life. Side effects can include dryness, burning, and congestion of the nasal mucosa, discomfort exhaling against the pressure, chest wall discomfort, middle ear discomfort, mask and machine noise, conjunctivitis from leaks into the eyes, and air swallowing. The incidence of side effects generally goes up with increased pressure. As such, a “pressure titration” is usually performed for a given patient to find a pressure that makes a reasonable trade-off between increasing effectiveness at eliminating respiratory related events and avoiding unpleasant side effects.

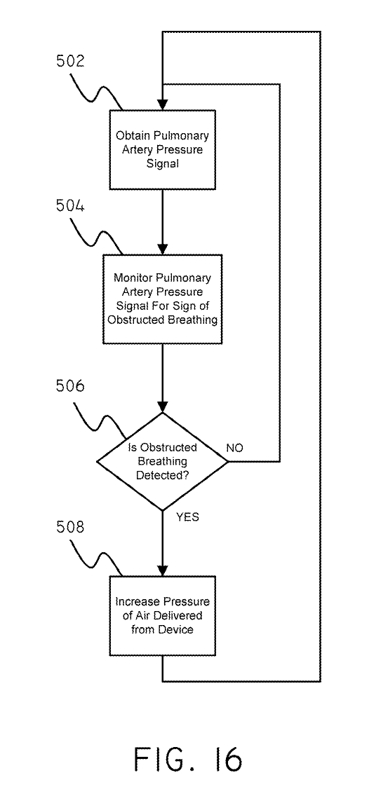

Embodiments of the present invention can include methods of automatically titrating the respiratory therapy delivered. For example, methods of the invention can include adjusting the pressure of air delivered during CPAP therapy based on pulmonary information as derived from a pulmonary artery pressure signal. In some embodiments, when breathing patterns are detected that indicate the upper airway is not sufficiently open, such as apnea or hypopnea, the pressure of air delivered during CPAP therapy is automatically increased. FIG. 16 shows a flowchart of an exemplary method of automatically adjusting the pressure of air delivered from an airway therapy device. A pulmonary artery pressure signal is obtained 502 from a pressure sensor that is disposed in or near the pulmonary artery. Next, the pulmonary artery pressure signal is monitored 504 for signs of obstructed breathing, such as apnea or hypopnea. A decision 506 is then made based on whether or not signs of obstructed breathing are detected. If obstructed breathing is not detected, then the process goes back to the step of obtaining 502 the pulmonary artery pressure signal. However, if obstructed breathing is detected, then the system increases 508 the pressure of air being delivered by the airway therapy device, before going back to the step of obtaining 502 the pulmonary artery pressure signal.

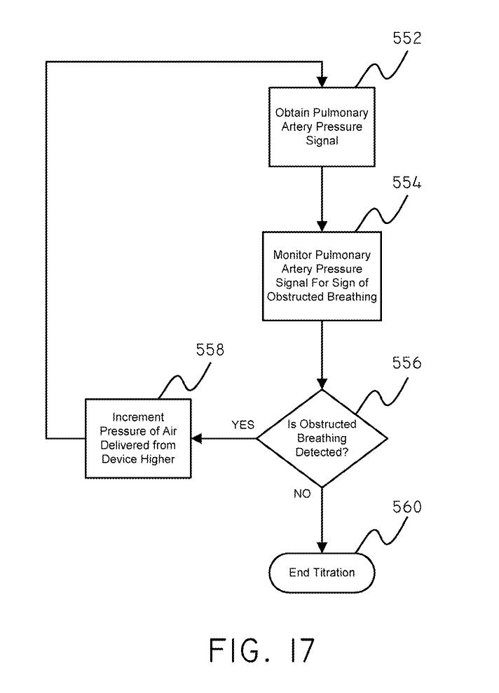

In some embodiments, titration can proceed by gradually increasing air pressure until symptoms, such as apnea or hypopnea, disappear. For example, referring now to FIG. 17, another embodiment of a method for titrating air pressure delivered by an airway therapy device is illustrated. A pulmonary artery pressure signal is obtained 552 from a pressure sensor that is disposed in or near the pulmonary artery. Next, the pulmonary artery pressure signal is monitored 554 for signs of obstructed breathing, such as apnea or hypopnea. A decision 556 is then made based on whether or not signs of obstructed breathing are detected. If obstructed breathing is detected, then the system increments 558 the pressure of air being delivered by the airway therapy device, before going back to the step of obtaining 552 the pulmonary artery pressure signal. However, if obstructed breathing is not detected, then the titration process is ended 560.

In other embodiments, titration can proceed by gradually decreasing air pressure until signs, such as apnea or hypopnea, appear. In some embodiments, titration can include both gradually increasing air pressure and gradually decreasing air pressure and monitoring for signs such as apnea or hypopnea in both circumstances.

A signal from a pulmonary artery pressure sensor can be processed by an implantable device and then information regarding the desired air pressure can be transmitted to a CPAP device. Alternatively, a signal from a pulmonary artery pressure sensor can be transmitted directly to a CPAP device which can process the pulmonary artery pressure signal in order to determine whether air pressure should be increased or not.

BiPAP is similar to CPAP but provides two levels of pressure, a higher pressure during inhalation and a lower pressure during exhalation. As such, methods of titration based on a pulmonary artery pressure signal as described above are also applicable in the context of BiPAP therapy.

Information about the cardiopulmonary status of a patient, can also be used to aid in the diagnosis and monitoring of sleeping disorders. Sleeping disorders are a significant problem affecting, by some estimates, almost 15% of the population. The broad category of sleep disorders can involve difficulties related to sleeping, including difficulty falling or staying asleep, falling asleep at inappropriate times, excessive total sleep time, or abnormal behaviors associated with sleep.

An exemplary sleeping disorder is sleep apnea. Sleep apneas are defined as conditions where breathing is interrupted by at least ten seconds during sleep. This can occur up to hundreds of times per night with incidence resulting in disturbed sleep. Sleep apneas can include both obstructive sleep apneas and central sleep apneas. Obstructive sleep apneas are where the interruption in breathing is caused by an airway obstruction. Central sleep apneas are where the interruption in breathing is caused by a problem with central nervous system control of breathing.

In an embodiment, the invention includes a method of detecting a sleeping disorder comprising measuring a pulmonary artery pressure signal with a pressure sensor; and monitoring the pulmonary artery pressure signal to identify a breathing pattern indicative of a sleeping disorder. Sleeping disorders that can be detected by can include sleep apneas, both obstructive sleep apneas and central sleep apneas. As described above with reference to FIG. 13, sleep apnea can be identified by a respiration signal reflecting the interruption of breathing for a threshold period of time. In some embodiment, the threshold period of time is equal to or greater than ten seconds. Each interruption to breathing can be recorded as it occurs so that the total number of interruptions (apneas) over a period of time can be accounted for. This running count of apneas occurring during sleeping hours can be stored and then transmitted to a care provider, such as through an advanced patient management system.

In some embodiments, data regarding sleeping disorders or disturbed breathing events such as apneas as detected through monitoring of a pulmonary artery pressure signal can be used in a closed loop system for controlling pacing therapy as delivered by an implantable cardiac rhythm management (CRM) device such as a pacemaker or another CRM device including pacing functions. While not intending to be bound by theory, it is believed that some types of sleep disorders can affect cardiac rhythm. Embodiments of the invention can include methods of controlling pacing therapy as delivered by an implantable CRM device in order to counteract changes to cardiac rhythm caused by a sleeping disorder or a disturbed breathing event. It is also believed that changes to cardiac pacing can act to ameliorate some sleeping disorders or reduce the incidence of disturbed breathing events, at least in some patients. For example, it is believed that increasing the pacing rate can have a positive effect on some patients with sleeping disorders or exhibiting disturbed breathing events. In an embodiment, the invention includes a method of providing closed loop therapy including monitoring a pulmonary artery pressure signal for changes indicative of a sleeping disorder or a disturbed breathing event and controlling pacing therapy parameters in a manner so as to respond to the sleeping disorder or disturbed breathing event. The term “closed loop”, as used herein, shall refer to a system in which therapy is regulated by system feedback without human intervention. Specifically, in some embodiments the pacing rate of a cardiac rhythm management (CRM) device can be increased in response to the detection of a sleeping disorder or a disturbed breathing event.

Information about the cardiopulmonary status of a patient, as gained through a pulmonary artery pressure signal, can be also be used to monitor sleeping habits, sleep quality, and/or sleep characteristics of patients. By way of example, a pulmonary artery pressure signal can be processed in order to derive information regarding the onset, termination, duration, stages, and quality of sleep experienced by a patient. Furthermore, this information can be trended over a period of time and can provide insight into the emotional and physical health of a patient.

The onset or termination of sleep can be manifested by various effects on cardiopulmonary parameters. By way of example, the onset or termination of sleep can affect heart rate, tidal volume, minute ventilation, blood pressure, and the like. A pulmonary artery pressure signal can be utilized to derive such cardiopulmonary parameters. Therefore, monitoring of a pulmonary artery pressure signal can be used to gather information regarding the occurrence or nature of a sleep event, such as the onset, termination, duration, stages, and quality of sleep experienced by a patient.

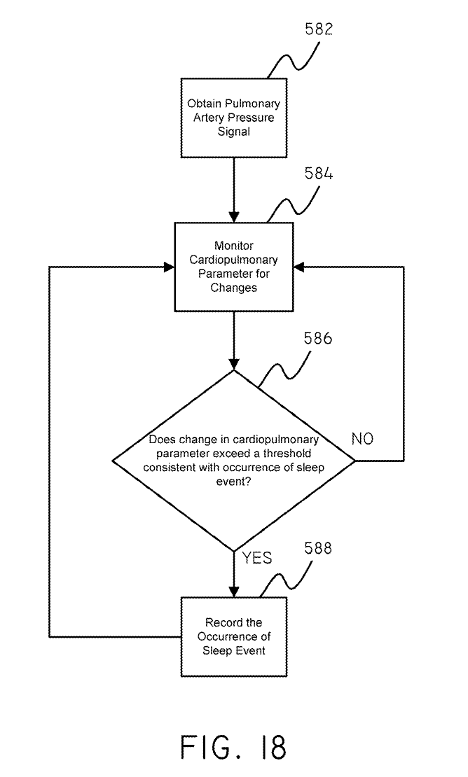

Referring now to FIG. 18, a flowchart of one method of monitoring the occurrence of a sleep event is illustrated. First, a pulmonary artery pressure signal is obtained 582 from a pressure sensor that is disposed in or near the pulmonary artery. Next, the pulmonary artery pressure signal is monitored 584 for changes to a cardiopulmonary parameter. A decision 586 is then made based on whether or not observed changes to the cardiopulmonary parameter are consistent with the occurrence of a sleep event. For example, the change to the cardiopulmonary parameter is evaluated to determine whether or not it exceeds a threshold amount. If the change in the cardiopulmonary parameter exceeds a threshold amount, the occurrence of a sleep event is recorded 588 before continuing to monitor 584 the cardiopulmonary parameter for further changes. However, if the change in the cardiopulmonary parameter fails to exceed a threshold amount, then monitoring 584 of the cardiopulmonary parameter is continued without recording the occurrence of a sleep event. The threshold amount can be set based on the desired sensitivity and accuracy and the individual history of the patient. In some cases, a calibration may be performed where the changes in the cardiopulmonary parameter associated with the occurrence of a sleep event for a given patient are noted and then the threshold values are set accordingly.

As a specific example, in some studies, heart rate has been found to decrease during the onset of sleep, attributed to a relative increase in parasympathetic tone. Heart rate can be derived from a pulmonary artery pressure signal as described above with reference to FIG. 3. In some embodiments, a reduction in heart rate beyond a threshold amount can be interpreted as an indicator of the onset of sleep.

As another specific example, in some studies, minute ventilation has been found to decrease by greater than 10% during sleep as a result of reduced tidal volume after the onset of sleep. Tidal volume and minute ventilation can be derived from a pulmonary artery pressure signal as outlined above. In some embodiments, a reduction in minute ventilation and/or a reduction in tidal volume beyond a threshold amount can be interpreted as an indicator of the onset of sleep.

For most patients, blood pressure decreases with the onset of sleep. In some embodiments, a reduction in blood pressure beyond a threshold amount can be interpreted as an indicator of the onset of sleep. In some embodiments, a reduction in pulmonary artery blood pressure beyond a threshold amount can be interpreted as an indicator of the onset of sleep.

In some embodiments, the onset or termination of sleep may be detected by combining data regarding a plurality of cardiopulmonary parameters as derived from a pulmonary artery signal. For example, in some embodiments, the reduction in pulmonary artery blood pressure beyond a threshold amount in combination with a reduction in minute ventilation and/or a reduction in tidal volume beyond a threshold amount is interpreted as an indicator of the onset of sleep.

In some embodiments, the onset or termination of sleep may be detected by combining data regarding cardiopulmonary parameters with other data or signals. By way of example, in some embodiments, sleep can be detected by combining information regarding cardiopulmonary parameters with information regarding a patient's posture, the time of day, accelerometer data, eye movement data, electroencephalogram (EEG) data, muscle tone data, body temperature data, pulse oximetry data, and the like.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A method of measuring a pulmonary function parameter of a patient comprising:
chronically implanting a pulmonary artery pressure sensor;
obtaining a pulmonary artery pressure signal from the pressure sensor; and
processing the signal to obtain the pulmonary function parameter.

2. The method of claim 1, comprising transmitting the pulmonary artery pressure signal to an implanted device.

3. The method of claim 2, the implanted device comprising a pulse generator.

4. The method of claim 2, comprising wirelessly transmitting the signal.

5. The method of claim 1, the pulmonary function parameter selected from the group consisting of respiratory rate, respiratory excursion, breath interval, inspiration slope, expiration slope, tidal volume, relative tidal volume, minute ventilation, relative minute ventilation, pulmonary vascular resistance, relative pulmonary vascular resistance, forced expiration volume in one second (FEV1), relative FEV1, forced vital capacity (FVC), relative FVC, total lung capacity (TLC) and relative (TLC).

6. The method of claim 1, wherein processing the signal to obtain the pulmonary function parameter includes the step of converting the pulmonary artery pressure signal into a respiration signal.

7. The method of claim 6, further comprising deriving the frequency of the respiration signal.

8. The method of claim 6, further comprising deriving the amplitude of the respiration signal.

9. The method of claim 1, further comprising comparing a real-time value of the pulmonary function parameter with a stored value of the pulmonary function parameter.

10. The method of claim 1, further comprising reporting the pulmonary function parameter to a care provider.

11. A method of detecting a sleeping disorder comprising:
measuring a pulmonary artery pressure signal with a pressure sensor; and
monitoring the pulmonary artery pressure signal to identify a breathing pattern indicative of a sleeping disorder.

12. The method of claim 11, wherein the pressure sensor is chronically implanted.

13. The method of claim 11, the pressure sensor disposed in the pulmonary artery of a patient.

14. The method of claim 11, the breathing pattern indicative of a central sleep apnea.

15. The method of claim 11, the breathing pattern indicative of an obstructive sleep apnea.

16. The method of claim 11, the breathing pattern characterized by a plurality of apneas.

17. The method of claim 11, the breathing pattern characterized by five or more obstructed breathing events per hour, the obstructed breathing event selected from the group consisting of an apnea and a hypopnea.

18. The method of claim 11, the breathing pattern characterized by a plurality of hypopneas.

19. The method of claim 11, the breathing pattern characterized by a plurality of cyclical increases and decreases in breathing amplitude.

20. A method of tracking sleep characteristics of a patient comprising:
monitoring a pulmonary artery pressure signal for a change to a cardiopulmonary parameter indicative of a sleep event; and
recording an occurrence of the sleep event if a change to the cardiopulmonary parameter indicative of the sleep event occurs.

21. The method of claim 20, further comprising evaluating the change to the cardiopulmonary parameter to determine if it exceeds a threshold value.

22. The method of claim 20, the sleep event comprising the onset or the cessation of sleep.

23. The method of claim 20, the cardiopulmonary parameter comprising a parameter selected from the group consisting of heart rate, tidal volume, minute ventilation, and blood pressure.

24. The method of claim 20, further comprising comparing changes of the cardiopulmonary parameter with additional data in order to determine whether or not a sleep event has occurred, the additional data selected from the group consisting of time, accelerometer data, posture, eye movement, temperature, blood oxygenation, and an electroencephalogram (EEG).
